(19) United States
(12) Patent Application Publication
(10) Pub. No.: US 2018/0055751 A1
(43) Pub. Date: Mar. 1, 2018

(54) COMPOSITIONS COMPRISING AN ASSOCIATON OF CATIONIC COMPOUNDS, SILANE COMPOUNDS, ESTERS AND FATTY SUBSTANCES

(71) Applicant: L'OREAL, PARIS (FR)

(72) Inventors: Halil GEVGILILI, Weehawken, NJ (US); Jun LIANG, Staten Island, NY (US)

(73) Assignee: L'OREAL, PARIS (FR)

(21) Appl. No.: 15/252,356

(22) Filed: Aug. 31, 2016

(51) Int. Cl. A61K 8/41; A61K 8/81; A61K 8/34; A61K 8/37; A61K 8/42; A61K 8/58; A61K 8/362; A61Q 5/02; A61Q 5/12; A61K 8/365; A61K 8/898

(52) U.S. Cl. CPC A61K 8/416; A61K 2800/596; A61K 8/342; A61K 8/375; A61K 8/42; A61K 8/585; A61K 8/362; A61Q 5/02; A61Q 5/12; A61K 8/365; A61K 8/898; A61K 8/345; A61K 2800/5426; A61K 2800/884; A61K 8/8176

(57) ABSTRACT

Disclosed are compositions for treating and conditioning keratinous substrates, comprising a first cationic surfactant chosen from alkyl quaternary ammonium or diammonium salts; a second cationic surfactant chosen from alkyl amines or alkyl amine salts; a first silane compound, a cationic vinylpyrrolidone polymer, fatty alcohols, glyceryl esters, organic acids, and water. Also disclosed are methods treating and conditioning keratinous substrates using the composition.

### FIELD OF THE INVENTION

The present application relates to cosmetic compositions for use on keratinous substrates, such as hair. In particular, it relates to compositions and methods for treating and conditioning hair.

### BACKGROUND

Certain hair types are naturally thin and/or fine. In addition, any type of hair can diminish in quality and/or quantity over time by age and/or due to factors such as natural greasiness, sweat, shredded skin cells from the scalp, pollution, and dirt. These factors can result in thinning hair and/or harm the visual appearance and the feel of the hair, and lead to lank body and decreased volume. The magnitude of the consequences of these factors, which are almost all inevitable, is variable, depending on, for example, the quality of the hair, length, style, and environmental factors.

Hair care products are used to combat these drawbacks. Conventional cleansing compositions such as shampoos, for example, which contain surfactants such as anionic, nonionic and/or amphoteric type surfactants, can be employed to remove the diverse types of soils typically present on the substrate such as hair.

These cleansing compositions, while providing good cleansing power, may yield poor intrinsic cosmetic properties due to the fact that the nature of such a cleansing treatment may result in a less conditioned or rough feel to the hair due to, for example, the gradual removal of the natural or applied fats, lipids, or proteins contained in or at the surface of the hair.

Thus, a second composition, a hair conditioner which is generally a rinse-off product, can be used on hair after it has been shampooed in order to confer conditioning, smoothing, and softening properties to the hair. Aside from such properties, it is highly desirable to design conditioners that can confer other cosmetic benefits such as those provided by hair styling products, for example gels and mousses that are leave-in compositions that impart volume and body while on the hair. Some leave-in styling products use polymers, for example film-forming polymers, to provide volumizing properties. However, some polymers can be easily removed from the hair, for example by rinsing or washing. Thus, any cosmetic benefits to the hair from such products are generally diminished or removed once the hair is rinsed or washed.

Thus the present disclosure relates to hair conditioning compositions. More particularly, the embodiments of the disclosure relate to rinse-off compositions that provide volumizing properties on keratinous substrates, such as keratin fibers, in particular hair, wherein the volumizing effects on hair can be long-lasting, that is, the effect remains even after repeated washings or cleansing using the compositions of the present disclosure. The disclosure also relates to a hair conditioning process using this composition.

It is also an object of embodiments of the disclosure to provide a stable rinse-off composition that conditions a keratinous substrate while imparting increased mass, body or volume and maintaining desirable or cosmetically acceptable deposition and film formation capability on hair fibers to provide excellent volumizing and cosmetic properties, for example long-lasting volume, conditioning, softness and detangling.

According to embodiments of the disclosure, a composition for treating and/or conditioning hair is provided, the composition comprising a first cationic surfactant chosen from alkyl quaternary ammonium or diammonium salts, a second cationic surfactant chosen from alkyl amines or alkyl amine salts, a silane compound, a cationic vinylpyrrolidone polymer, fatty alcohols, a glyceryl ester, organic acids, and water. It has now been surprisingly and unexpectedly discovered that such a composition is stable, and delivers instant as well as long lasting mass, body, and/or volume to hair. Hair treated and/or contacted with the compositions according to embodiments of the disclosure was found to have improved mass, body, volume, a fuller appearance, and easy to style or shape as well as easy to comb/detangle while wet. At the same time, the hair is sufficiently conditioned.

Also disclosed is a process for imparting conditioning and other cosmetic properties to keratinous substrates such as the hair and/or the scalp using the composition according to embodiments of the disclosure, the process including applying to the keratinous substrates and/or scalp, a composition as defined above, rinsing off the composition, and optionally drying said keratinous substrates.

Other subjects, characteristics, aspects and advantages of embodiments of the disclosure will emerge even more clearly on reading the description and the various examples that follow.

### BRIEF SUMMARY

Embodiments of the disclosure relate to a composition for treating keratinous substrates, the composition comprising:
(a) at least one chosen from alkyl quaternary ammonium or diammonium salts, and mixtures thereof;
(b) at least one a second cationic surfactant chosen from alkyl amines, alkyl amine salts, and mixtures thereof;
(c) a first silane compound corresponding to formula (I):

R₁Si(OR₂)_{z}(R₃)ₓ(OH)_{y} (I)
in which:
   R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
   R₂ and R₃, which may be identical or different, represent a linear or branched alkyl group comprising from 1 to 6 carbon atoms,
   y denotes an integer ranging from 0 to 3, and
   z denotes an integer ranging from 0 to 3, and
   x denotes an integer ranging from 0 to 2, with z+x+y=3;
(d) at least one cationic vinylpyrrolidone polymer;
(e) two or more fatty alcohols;
(f) at least one glyceryl ester;
(g) organic acids; and
(h) water.

Embodiments of the disclosure also relate to a process for conditioning a keratinous substrate, such as hair and/or the scalp, involving applying the above-described composition onto the keratinous substrate, and to methods of increasing the volume of the keratinous substrate such as hair by treating or contacting with the hair above-described composition.

The compositions of embodiments of the disclosure are stable over time and do not undergo phase separation. In addition, the compositions are creamy in appearance.

It is to be understood that both the foregoing general description and the following detailed description are exemplary and explanatory only, and are not restrictive of the disclosure.

### DETAILED DESCRIPTION

As used herein, the expression “at least one” means one or more and thus includes individual components as well as mixtures/combinations.

In the present patent application, a species is termed as being “cationic” when it bears at least one permanent positive charge or when it can be ionized as a positively charged species, under the conditions of use of the compositions of embodiments of the disclosure (for example the medium or the pH) and not comprising any anionic filler.

A species is termed as being “nonionic” when it is neither cationic nor anionic within the meaning of the disclosure, in particular when it comprises no cationic or anionic groups within the meaning of the disclosure.

A species is termed as being “anionic” when it bears at least one permanent negative charge or when it can be ionized as a negatively charged species, under the conditions of use of the compositions of embodiments of the disclosure (for example the medium or the pH) and not comprising any cationic filler.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%), such as within 9%, 8%, 7%, 6%, 5%, 4%, 3%, 2%, or 1%, according to various embodiments.

The term “comprising” (and its grammatical variations) as used herein is used in the inclusive sense of “having” or “including” and not in the exclusive sense of “consisting only of”.

“Keratinous substrates” as used herein, includes, but is not limited to keratin fibers such as hair on the human head and hair comprising eyelashes. “Keratinous substrates” as used herein, may also refer to the skin such as lips, finger nails or toe nails, and the scalp.

As used herein, the terms “applying a composition onto “keratinous substrates” as used herein, includes, and “applying a composition onto “keratinous substrates” or “keratin fibers” such as hair on a human head with at least one of the compositions of the disclosure, in any manner.

As used herein, “formed from,” means obtained from chemical reaction of, wherein “chemical reaction,” includes spontaneous chemical reactions and induced chemical reactions. As used herein, the phrase “formed from,” is open ended and does not limit the components of the composition to those listed.

The term “stable” as used herein means that the composition does not exhibit phase separation and/or crystallization.

The term “treat” (and its grammatical variations) as used herein refers to the application of the compositions of the present disclosure onto keratinous substrates such as hair. The term “treat” (and its grammatical variations) as used herein also refers to contacting keratinous substrates such as hair with the compositions of the present disclosure onto

“Volatile”, as used herein, means having a flash point of less than about 100° C.

“Non-volatile”, as used herein, means having a flash point of greater than about 100° C.

“Substituted” as used herein, means comprising at least one substituent.

Non-limiting examples of substituents include atoms, such as oxygen atoms and nitrogen atoms, as well as functional groups, such as acyloxyalky groups, carboxylic acid groups, amine or amino groups, acylamino groups, amide groups, halogen containing groups, ester groups, thiol groups, sulphonate groups, thiosulphate groups, siloxane groups, and polysiloxane groups. The substituent(s) may be further substituted.

The terms “organic compound” and “having an organic structure” mean compounds containing carbon atoms and hydrogen atoms and optionally heteroatoms such as S, O, N or P, alone or in combination.

The term “polymer” is understood to mean, within the meaning of the disclosure, a compound characterized by the multiple repetition of one or more species of atoms or groups of atoms, known as monomers, linked to each other in amounts sufficient to provide a set of properties that do not vary markedly with the addition or removal of one or a few of the monomers.

The term “film-forming polymer” is understood to mean a polymer which is capable of forming, by itself alone or in the presence of an additional film-forming agent, a macroscopically continuous or semi-continuous film on a support, in particular on keratinous substances, such as a cohesive film.

The term “rinse-off” is used herein to mean that a keratinous substrate such as hair is rinsed and/or washed with water either after or during the application of a composition onto the keratinous substrate, and before drying and/or styling said keratinous substrate. At least a portion of the composition is removed from the keratinous substrate during the rinsing and/or washing. A “rinse-off” product refers to a composition such as a hair care composition that is rinsed and/or washed with water either after or during the application of the composition onto the keratinous substrate, and before drying and/or styling said keratinous substrate.

The compositions and methods of the present disclosure can comprise, consist of, or consist essentially of the essential elements and limitations of the disclosure described herein, as well as any additional or optional ingredients, components, or limitations described herein or otherwise useful.

An embodiment of the present disclosure is directed to a composition for treating keratinous substrates, the composition comprising:
(a) at least one first cationic surfactant chosen from alkyl quaternary ammonium or diammonium salts, and mixtures thereof;
(b) at least one second cationic surfactant chosen from alkyl amines, alkyl amine salts, and mixtures thereof;
(c) a first silane compound corresponding to formula (I):

R₁Si(OR₂)_{z}(R₃)ₓ(OH)_{y} (I)
in which:
   R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
   R₂ and R₃, which may be identical or different, represent a linear or branched alkyl group comprising from 1 to 6 carbon atoms,
   y denotes an integer ranging from 0 to 3, and
   z denotes an integer ranging from 0 to 3, and
   x denotes an integer ranging from 0 to 2, with z+x+y=3;
(d) at least one cationic vinylpyrrolidone polymer;
(e) two or more fatty alcohols;
(f) at least one glyceryl ester;
(g) organic acids; and
(h) water.

In one embodiment, the first cationic surfactant is chosen from alkyl quaternary ammonium salts.

In one embodiment, the first cationic surfactant is chosen from behentrimonium chloride, cetrimonium chloride, quaternium-22, behenylamidopropyl-2, 3-di-hydroxypropyldimethylammonium chloride, palmitylamidopropyltrimethylammonium chloride, and chloride and methyl sulfate of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, distearyldimethylammonium chloride, oleocetyldimethylhydroxyethylammonium chloride, stearamidopropyldimethyl (myristyl acetate) ammonium chloride, di(C1-C2 alkyl) (C12-C22 alkyl)hydroxy(C1-C2alkyl)ammonium salt, or alkyltrimethylammonium salt in which the alkyl radical comprises 12 to 24 carbon atoms, propanetallowdiammonium dichloride, behentrimonium methosulfate, quaternium-83, quaternium-87, and mixtures thereof.

In one embodiment, the first cationic surfactant is chosen from behentrimonium chloride, cetrimonium chloride, behentrimonium methosulfate, and mixtures thereof.

In one embodiment, the amount of the first cationic surfactant ranges from about 0.1% to about 10% by weight, based on the total weight of the composition.

In one embodiment, the second cationic surfactant is chosen from dimethylamine derivatives.

In one embodiment, the dimethylamine derivative is chosen from stearyl dimethyl amine, stearamidopropyl dimethylamine, brassicamidopropyl dimethylamine, and mixtures thereof.

In one embodiment, the second cationic surfactant is stearamidopropyl dimethylamine.

In one embodiment, the amount of the second cationic surfactant ranges from about 0.05% to about 5% by weight, based on the total weight of the composition.

In one embodiment, the first silane compound (c) of formula (I) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, their oligomers, and mixtures thereof.

In one embodiment, the first silane compound (c) of formula (I) is chosen from 3-aminopropyltriethoxysilane, oligomers thereof, and mixtures thereof.

In one embodiment, the amount of the first silane compound (c) of formula (I) ranges from about 0.05% to about 5% by weight, based on the total weight of the composition.

In one embodiment, the cationic vinylpyrrolidone polymer is chosen from vinylpyrrolidone/dimethylaminoethyl methacrylate copolymers quaternized with diethyl sulfate, polymeric quaternary ammonium salt consisting of vinylpyrrolidone and dimethylaminopropyl methacrylamide monomers, and mixtures thereof.

In one embodiment, the cationic vinylpyrrolidone polymer is chosen from polyquaternium-11, polyquaternium-28, and mixtures thereof.

In one embodiment, the amount of the cationic vinylpyrrolidone polymer ranges from about 0.05% to about 5% by weight, based on the total weight of the composition.

In one embodiment, the two or more fatty alcohols are chosen from non-alkoxylated, saturated or unsaturated, linear or branched fatty alcohols having from 6 to 60 carbon atoms.

In one embodiment, the two or more fatty alcohols are chosen from cetyl alcohol, stearyl alcohol, cetearyl alcohol (mixture of cetyl alcohol and stearyl alcohol), octyldodecanol, 2-butyloctanol, 2-hexyldecanol, 2-undecylpentadecanol, oleic alcohol, linoleic alcohol, behenyl alcohol, 2-dodecylhexadecanol, 2-tetradecyl-1-octadecanol, 2-tetradecyl-1-eicosanol, 2-hexadecyl-1-octadecanol and 2-hexadecyl-1-eicosanol, octyldodecanol, 2-butyloctanol, 2-hexyldecanol, 2-undecylpentadecanol, oleyl alcohol or linoleyl alcohol, isostearyl alcohol, and mixtures thereof.

In one embodiment, the composition comprises fatty alcohols chosen from cetearyl alcohol (a mixture of two fatty alcohols, cetyl alcohol and stearyl alcohol).

In one embodiment, the composition comprises cetyl alcohol and cetearyl alcohol.

In one embodiment, the composition comprises cetyl alcohol and stearyl alcohol.

In one embodiment, the fattyl alcohols of the present invention are chosen from cetyl alcohol, stearyl alcohol, cetearyl alcohol, and mixtures thereof.

In one embodiment, the amount of the fatty alcohols ranges from about 1% to about 15% by weight, based on the total weight of the composition.

In one embodiment, the glyceryl esters of the present invention include but are not limited to, glyceryl monoesters, such as glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof.

In one embodiment, the glyceryl ester is chosen from glyceryl oleate, glyceryl monostearate (glyceryl stearate), glyceryl monoisostearate, glyceryl monopalmitate, glyceryl monobehenate, and mixtures thereof.

In one embodiment, the glyceryl ester is chosen from glyceryl stearate.

In one embodiment, the amount of the glyceryl ester ranges from about 0.05% to about 5% by weight, based on the total weight of the composition.

In one embodiment, the organic acids are chosen from mono-carboxylic acids and polycarboxylic acids (with 2 or more carboxylic acid groups).

In one embodiment, the organic acids are chosen from acetic acid, terephthalic acid, HOOC-PEG-COOH acid; citric acid, tartaric acid; betaine hydrochloride, gluconic acid or 2-ethylcaproic acid, lactic acid, salicylic acid, glycolic acid, malic acid, maleic acid, oxalic acid, malonic acid, aspartic acid, glutamic acid, benzoic acid, acetic acid, formic acid, aspartic acid, glutamic acid, oxalic acid, succinic acid, mucic acid, phthalic acid, poly(ethylene glycol) bis(carboxymethyl)ethers, acrylic polyacid, copolymer of acrylic acid and maleic acid, polyaspartic acid, and carboxylic polydimethylsiloxanes, sulfonic acids such as benzene sulfonic acid, sulfonic acid HSO2OH, taurine, 2-[4-(2-hydroxy-ethyl)-piperazin-1-yl]ethane sulfonic acid (or HEPES), and (3E)-3-(4-{(E)-[7,7-dimethyl-3-oxo-4-(sulfomethyl)bicyclo[2.2.1]hept-2-ylidene]methyl}benzylidene)-7,7-dimethyl-2-oxobicyclo[2.2.1]hept-1-yl]methane sulfonic acid, trichloroacetic acid, L-poly(ethylene glycol) bis(carboxymethyl)ether having a molecular weight of 250 g/mol, salicylic acid derivatives, jasmonic acid derivative, 3-hydroxy-2-pentyl-cyclopentyl acetic acid, 2-[4-(2-hydroxy-ethyl)-piperazin-1-yl]-ethanesulfonic acid, pyruvic acid, mandelic acid, and mixtures thereof.

In one embodiment, the organic acids are chosen from tartaric acid, lactic acid, malic acid, maleic acid, oxalic acid, malonic acid, citric acid, aspartic acid, salicylic acid, benzoic acid, acetic acid, formic acid, and mixtures thereof.

In one embodiment, the organic acids comprise tartaric acid and lactic acid.

In one embodiment, the tartaric acid is present an amount such that the ratio by weight of the second cationic surfactant to tartaric acid ranges from about 6 to about 2 or about 5 to about 2.5 or about 4.3 to about 2.8, and is preferably at about 3.8.

In one embodiment, the lactic acid is present an amount such that the ratio by weight of APTES to lactic acid ranges from about 3 to about 0.5 or about 2 to about 0.6 or about 1.7 to about 0.9, and is preferably at about 1.4.

In one embodiment, the composition further comprises a second silane compound other than the first silane (c) and chosen from dimethicone, amino silicones comprising at least one primary, secondary or tertiary amine or a quaternary ammonium group, and mixtures thereof.

In one embodiment, the second silane compound is an amino silicone chosen from amodimethicone, bis-isobutyl/PEG/PPG-20/35/amodimethicone copolymer, bis-cetearyl amodimethicone, bis-amino PEG/PPG-41/3 aminoethyl PG-propyl dimethicone, PEG-40/PPG-8 methylaminopropyl hydroxypropyl dimethicone copolymer, bis-isobutyl/PEG/PPG-20/35/amodimethicone copolymer, and mixtures thereof.

In one embodiment, the second silane compound is chosen from amodimethicone.

In one embodiment, the amount of the second silane compound ranges from about 0.04% to 1% by weight, based on the total weight of the composition.

In one embodiment, the amino silicone is provided as an emulsion comprising at least one surfactant selected from a nonionic surfactant, a cationic surfactant, and mixtures thereof.

In one embodiment, the keratinous substrates include hair.

In one embodiment, the composition is a rinse-off conditioning composition.

In an embodiment of the present disclosure, the composition is a rinse-off composition for conditioning keratinous substrates, in particular hair, wherein the composition comprises:
(a) from about 0.2% to about 8% by weight of at least one first cationic surfactant chosen from alkyl quaternary ammonium or diammonium salts, and mixtures thereof;
(b) from about 0.1% to about 4.5% by weight of at least one second cationic surfactant chosen from dimethylamine derivatives;
(c) from about 0.1% to about 4% by weight of a first silane compound corresponding to formula (I):

R₁Si(OR₂)_{z}(R₃)ₓ(OH)_{y} (I)
in which:
   R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
   R₂ and R₃, which may be identical or different, represent a linear or branched alkyl group comprising from 1 to 6 carbon atoms,
   y denotes an integer ranging from 0 to 3, and
   z denotes an integer ranging from 0 to 3, and
   x denotes an integer ranging from 0 to 2, with z+x+y=3;
(d) from about 0.05% to about 4% by weight of at least one cationic vinylpyrrolidone polymer chosen from vinylpyrrolidone/dimethylaminoethyl methacrylate copolymers quaternized with diethyl sulfate, polymeric quaternary ammonium salt consisting of vinylpyrrolidone and dimethylaminopropyl methacrylamide monomers, and mixtures thereof;
(e) from about 1.2% to about 10% by weight of fatty alcohols chosen from cetyl alcohol, stearyl alcohol, cetearyl alcohol, and mixtures thereof;
(f) from about 0.1% to about 1.5% by weight of at least one glyceryl ester;
(g) organic acids comprising lactic acid and tartaric acid; and
(h) water;
   all weights being based on the total weight of the composition;
   wherein the ratio by weight of the second cationic surfactant to tartaric acid ranges from about 6 to about 2 or from about 5 to about 2.5 or from about 4.3 to about 2.8, and preferably, is at about 3.8; and
   wherein the ratio by weight of APTES to lactic acid ranges from about 3 to about 0.5 or from about 2 to about 0.6 or from about 1.7 to about 0.9, and preferably, is at about 1.4.

In one embodiment, a method for treating and/or conditioning a keratinous substrate, comprises contacting the keratinous substrate such as hair with the above-described compositions.

In one embodiment, the method comprises a step of leaving the composition on the keratinous substrate for a leave-on time and followed by a step of rinsing the keratinous substrate with water.

In one embodiment, the composition of the present disclosure is capable of being applied onto the keratinous substrates following the application of a shampoo or a cleansing or a detergent-based composition onto the keratinous substrates.

In another embodiment of the present disclosure, the composition is a rinse-off composition for conditioning keratinous substrates, in particular hair, wherein the composition comprises:
(a) from about 0.8% to about 3% by weight of at least one first cationic surfactant chosen from behentrimonium chloride, cetrimonium chloride, behentrimonium methosulfate, and mixtures thereof;
(b) from about 0.3% to about 2% by weight of at least one second cationic surfactant chosen from stearyl dimethyl amine, stearamidopropyl dimethylamine, brassicamidopropyl dimethylamine, and mixtures thereof;
(c) from about 0.4% to about 0.8% by weight of a first silane compound chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, their oligomers, and mixtures thereof;
(d) from about 0.05% to about 0.5% by weight of at least one cationic vinylpyrrolidone polymer chosen from polyquaternium-11, polyquaternium-28, and mixtures thereof;
(e) from about 1.5% to about 5% by weight of fatty alcohols chosen from cetyl alcohol, stearyl alcohol, cetearyl alcohol, and mixtures thereof
(f) from about 0.2% to about 2% by weight of at least one glyceryl ester chosen from glyceryl oleate, glyceryl monostearate (glyceryl stearate), glyceryl monoisostearate, glyceryl monopalmitate, glyceryl monobehenate, and mixtures thereof;
(g) organic acids comprising lactic acid and tartaric acid; and
(h) water;
   all weights being based on the total weight of the composition;
   wherein the ratio by weight of the second cationic surfactant to tartaric acid ranges from about 4.3 to about 2.8; and
   wherein the ratio by weight of APTES to lactic acid ranges from about 1.7 to about 0.9.

The compositions according to various embodiments of the disclosure have a homogenous texture, i.e., not lumpy, and are easy to apply and spread on the hair.

It has been surprisingly and unexpectedly discovered that the compositions according to the disclosure are stable over time, exhibit no visible phase separation, and allow retention of the cosmetic effects of the cationic surfactants, the cationic vinylpyrrolidone polymer, the first silane compound, the fatty alcohols and glyceryl esters such that hair is effectively or satisfactorily volumized and easy to detangle and comb through after treatment with the composition. It is possible that the volumizing effect imparted to the hair remains even after several washings of the hair.

### First Cationic Surfactants

The first cationic surfactants of the present disclosure may be chosen from alkyl quaternary ammonium or diammonium salts, and mixtures thereof.

Suitable examples of quaternary ammonium compounds are chosen from compounds, including their salts, of the general formula (A) below:

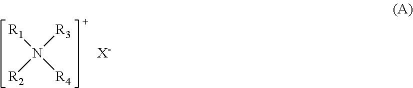

wherein R1, R2, R3, and R4, may independently be chosen from saturated or unsaturated, linear or branched, aliphatic hydrocarbon radicals comprising from 1 to about 30 carbon atoms, or an alkoxy, alkoxycarbonylalkyl, polyoxyalkylene, alkylamidoalkyl, hydroxyalkyl, aromatic, aryl or alkylaryl radical comprising from about 12 to about 30 carbon atoms, with at least one radical among R1, R2, R3 and R4 denoting a radical comprising from 8 to 30 carbon atom; and X″″ is chosen from halides, phosphates, acetates, lactates, (C2-C6) alkyl sulfates, and alkyl- or alkylaryl-sulfonates.

The quaternary ammonium compound (A) may be selected from behenyltrimethylammonium chloride (also called behentrimonium chloride), cetyltrimethylammonium chloride (also called cetrimonium chloride), quaternium-22, behenylamidopropyl-2, 3-di-hydroxypropyldimethylammonium chloride, palmitylamidopropyltrimethylammonium chloride, and chloride and methyl sulfate of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, and mixtures thereof.

Other suitable examples of quaternary ammonium compounds include distearyldimethylammonium chloride, oleocetyldimethylhydroxyethylammonium chloride, stearamidopropyldimethyl (myristyl acetate) ammonium chloride, di(C1-C2 alkyl) (C12-C22 alkyl)hydroxy(C1-C2alkyl)ammonium salt, such as dialkyldimethylammonium or alkyltrimethylammonium salt in which the alkyl radical preferably comprises 12 to 24 carbon atoms, propanetallowdiammonium dichloride, behentrimonium methosulfate, and mixtures thereof.

Non-limiting examples of quaternary ammonium salts that comprise the first cationic surfactants of the present disclosure include in particular behentrimonium chloride, cetrimonium chloride, behentrimonium methosulfate, and mixtures thereof.

A preferred quaternary ammonium compound (A) is behentrimonium chloride (INCI name) sold under the tradename VARISOFT BT 85 by Evonik Goldschmidt.

In certain embodiments, the total amount of the first cationic surfactant ranges from about 0.1% to about 10% by weight, such as from about 0.2% to about 8% by weight, from about 0.5% to about 6% by weight, from about 0.6% to about 5% by weight, or from about 0.8% to about 3%, by weight of active material, based on the total weight of the composition of the present disclosure, including all ranges and subranges therebetween.

In various embodiments, the total amount of the first cationic surfactants is about 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.25%, 2.5%, 2.75%, 3%, 3.25%, 3.5%, 3.75%, 4%, 4.25%, 4.5%, 4.75%, 5%, 5.5%, 6%, 6.5%, 7%, 7.5%, 8%, 9%, and 10%, by weight of active material, based on the total weight of the composition of the present disclosure.

### Second Cationic Surfactant

The composition of the invention comprises at least one second cationic surfactant. Non-limiting examples of second cationic surfactants that can be used in the current compositions include dimethylamine derivatives, such as for example stearyl dimethyl amine, stearamidoproppyl dimethylamine, brassicamidopropyl dimethylamine, and mixtures thereof.

In an embodiment the second cationic surfactant is selected from stearamidopropyl dimethylamine.

A particularly preferred second cationic surfactant for use in the present invention is stearamidopropyl dimethylamine, commercially available under the tradename MACKINE 301, from Rhodia.

The compositions of the disclosure preferably contain the second cationic surfactant in an amount of from about 0.05% to about 5% by weight, such as from about 0.1% to about 4.5% by weight, from about 0.2% to about 4% by weight, from about 0.25% to about 3% by weight, or from about 0.3% to about 2% by weight, or from about 0.3% to about 1% by weight of active material, based on the total weight of the composition, including all ranges and subranges therebetween.

In various embodiments, the total amount of the second cationic surfactant is about 0.1%, 0.125%, 0.15%, 0.175%, 0.2%, 0.25%, 0.3%, 0.33%, 0.35%, 0.375%, 0.4%, 0.425%, 0.45%, 0.475%, 0.5%, 0.525%, 0.55%, 0.575%, 0.6%, 0.625%, 0.65%, 0.675%, 0.7%, 0.725%, 0.75%, 0.775%, 0.8%, 0.825%, 0.83%, 0.85%, 0.875%, 0.9%, 0.925%, 0.95%, 0.975%, 1%, 1.25%, 1.5%, 1.75%, 2%, 2.5%, 3%, 3.5%, 4%, 4.5%, and 5%, by weight of active material, based on the total weight of the composition.

In an embodiment, the compositions of the present disclosure comprise a first cationic surfactant selected from behentrimonium chloride and a second cationic surfactant selected from stearamidopropyl dimethylamine.

### First Silane Compound

According to the invention, the composition comprises a first silane compound corresponding to formula (I) and/or their oligomers thereof.

Formula (I) is as follows:

R₁Si(OR₂)_{z}(R₃)ₓ(OH)_{y} (I)

in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C1-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3.

The term “oligomer” means the polymerization products of the compounds of formula (I) comprising from 2 to 10 silicon atoms.

Preferably, R₁ is a linear or branched, preferably linear, saturated C₁-C₂₂, in particular C₂-C₁₂, hydrocarbon-based chain, which may be substituted with an amine group NH₂ or NHR (R=C₁-C₂₀, in particular C₁-C₆, alkyl).

Preferably, R₂ represents an alkyl group comprising from 1 to 4 carbon atoms, better still a linear alkyl group comprising from 1 to 4 carbon atoms, and preferably the ethyl group.

Preferably, z ranges from 1 to 3.

Preferably, y=0.

Preferentially, z=3, and therefore x=y=0.

In one embodiment of the invention, R₁ represents a linear alkyl group comprising from 7 to 18 carbon atoms and more particularly from 7 to 12 carbon atoms, or a C1-C₆, preferably C₂-C₄, aminoalkyl group. More particularly, R₁ represents an octyl group.

In one embodiment of the invention, R₁ is a linear or branched, saturated or unsaturated C₁-C₂₂ hydrocarbon-based chain, substituted with an amine group NH₂ or NHR (R=C₁-C₂₀, in particular C₁-C₆, alkyl, C3-C₄₀ cycloalkyl or C₆-C₃₀ aromatic). In this variant, R₁ preferably represents a C1-C₆, preferably C₂-C₄, aminoalkyl group.

Preferably, the first silane compound of the present invention is an alkoxysilane.

Preferably, the composition comprises at least one compound of formula (Ib) chosen from alkoxysilanes such as 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, and mixtures thereof.

In certain embodiments, the siliane of the disclosure is an alkoxysilane selected from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane or oligomers thereof, and mixtures thereof.

In preferred embodiments, the first silane compound of the present invention is chosen from 3-aminopropyltriethoxysilane (APTES) or oligomers thereof, or mixtures thereof.

The first silane compound used in the composition of the invention, in particular those comprising a basic function, may be partially or totally neutralised in order to improve the water-solubility thereof. In particular, the neutralising agent may be chosen from organic or inorganic acids, such as citric acid, tartaric acid, lactic acid or hydrochloric acid, preferably, lactic acid.

Preferably, the optionally neutralised silanes according to the invention are water-soluble and in particular soluble at a concentration of 2%, better still at a concentration of 5% and even better still at a concentration of 10% by weight in water at a temperature of 25° C. and at atmospheric pressure (1 atm). The term “soluble” is intended to mean the formation of a single macroscopic phase.

The silane(s) of formula (I) and/or oligomers thereof may be present in the composition according to the invention in an amount of about 0.05% to about 5% by weight, such as from about 0.1% to about 4% by weight, from about 0.2% to about 3% by weight, from about 0.3% to about 2% by weight, from about 0.4% to about 1%, or from about 0.4% to less than 1%, such as from about 0.4% to about 0.8%, by weight of active material, based on the total weight of the composition, including all ranges and subranges therebetween.

In various embodiments, the total amount of the silane(s) of formula (I) is about 0.05%, 0.075%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.25%, 1.5%, 1.75%, 2%, 2.25%, 2.5%, 2.75%, 3%, 3.5%, 4%, 4.5%, 5% by weight of active material, based on the total weight of the composition.

### Cationic Vinylpyrrolidone Polymer

The at least one cationic vinylpyrrolidone polymer of the present disclosure may be chosen from vinylpyrrolidone/dimethylaminoethyl methacrylate copolymers quaternized with diethyl sulfate, polymeric quaternary ammonium salt consisting of vinylpyrrolidone and dimethylaminopropyl methacrylamide monomers, and mixtures thereof.

In some embodiments, the cationic vinylpyrrolidone polymer of the present disclosure is chosen from vinylpyrrolidone/dimethylaminoethyl methacrylate copolymers quaternized with diethyl sulfate such as polyquaternium-11 (INCI name), commercially available from A & E Connock (Perfumery & Cosmetics) or under the tradenames DEHYQUART CC 11 OR LUVIQUAT PQ 11 PN (BASF Corporation) or GAFQUAT 440 OR GAFQUAT 734 OR GAFQUAT 755 OR GAFQUAT 755N (Ashland Inc.) or ORISTAR PQ11 (Orient Stars LLC) or POLYQUAT-11 SL (Sino Lion (USA) Ltd.) or TRIquat 11 N-CC (TRI-K Industries, Inc., a Member of the Galaxy Group)

In other embodiments, the cationic vinylpyrrolidone polymer of the present disclosure is chosen from a polymeric quaternary ammonium salt consisting of vinylpyrrolidone and dimethylaminopropyl methacrylamide monomers such as polyquaternium-28 (INCI name) commercially available from A & E Connock (Perfumery & Cosmetics) or under the tradenames CONDITIONEZE NT-20 (Ashland Inc.) OR GAFQUAT HS-100 (Ashland Inc.).

The cationic vinylpyrrolidone polymer may be present in the composition according to the invention in an amount of from about 0.05% to 5% by weight, such as from about 0.075% to about 4% by weight, from about 0.1% to about 3% by weight, from about 0.1% to about 2% by weight, from about 0.1% to about 1%, or from about 0.05% to less than 1% by weight, such as from about 0.05% to about 0.8% by weight or such as from about 0.05% to about 0.5% by weight or such as from about 0.05% to about 0.25% by weight of active material, based on the total weight of the composition, including all ranges and subranges therebetween.

In various embodiments, the total amount of the cationic vinylpyrrolidone polymer is about 0.05%, 0.075%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.25%, 1.5%, 1.75%, 2%, 2.25%, 2.5%, 2.75%, 3%, 3.5%, 4%, 4.5%, 5% by weight of active material, based on the total weight of the composition.

### Fatty Alcohol

The compositions of the present disclosure may further comprise at least one fatty alcohol. The fatty alcohols that may be used in the composition of the disclosure include, but are not limited to, non-alkoxylated, saturated or unsaturated, linear or branched, and have from 6 to 60 carbon atoms, such as from 8 to 30 carbon atoms.

The fatty alcohols of the present disclosure are chosen from solid and liquid fatty alcohols.

The saturated liquid fatty alcohols can be branched. They can optionally comprise, in their structure, at least one aromatic or non-aromatic ring. They can be acyclic.

The unsaturated liquid fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They can be acyclic. Among the liquid unsaturated fatty alcohols, oleyl alcohol, linoleyl alcohol, linolenyl alcohol and undecylenyl alcohol may be mentioned.

Liquid fatty alcohols may be selected, for example, from octyldodecanol, 2-butyloctanol, 2-hexyldecanol, 2-undecylpentadecanol, oleyl alcohol or linoleyl alcohol, isostearyl alcohol, and mixtures thereof.

Solid fatty alcohols may be crystalline, amorphous or pasty. The solid fatty alcohols of the present invention are solid at room temperature (25 degrees centigrade) and at atmospheric pressure (1 atm) and are insoluble in water (i.e. they have a solubility in water of less than 1% by weight and preferably less than 0.5% by weight, at 25° C. and 1 atm) and are soluble, under the same temperature and pressure conditions, in at least one organic solvent (for example ethanol, chloroform, benzene or liquid petroleum jelly) to at least 1% by weight.

In an embodiment, the solid fatty alcohols preferably have a melting point of greater than or equal to 28° C. and have a viscosity, at a temperature of 40° C. and at a shear rate of 1 s⁻¹, of greater than or equal to 1 Pa·s.

In an embodiment, the melting point of the fatty alcohols ranges from 30° C. to 250° C., such as from 32° C. to 150° C. or such as from 35° C. to 150° C.

The melting points may be measured by DSC or on a Kofler bench. The melting point may be measured by differential calorimetric analysis (DSC) with a temperature rise of 10° C. per minute. The melting point is then the temperature corresponding to the top of the melting endotherm peak obtained during the measurement.

The viscosity measurements may be taken at a temperature of about 40° C. using an RS600 rheometer from Thermoelectron.

The solid fatty alcohols of the present invention are chosen from saturated or unsaturated, linear or branched, preferably linear and saturated, (mono) alcohols comprising from 6 to 60 carbon atoms, such as from 10 to 50 carbon atoms, or such as from 12 to 24 carbon atoms.

The solid fatty alcohols preferably have the structure of formula: R—OH in which R especially denotes a C6-C60, for example, C8-C60, preferably C10-C50 or even C12-C30 alkyl group, R possibly being substituted with one or more hydroxyl groups, R possibly being branched. The solid fatty alcohols of the invention may be non-oxyalkylenated and/or non-glycerolated. These fatty alcohols may be constituents of animal or plant waxes.

The solid fatty alcohol may represent a mixture of fatty alcohols, which means that several species of fatty alcohol may coexist, in the form of a mixture, in a commercial product. One example of such a commercial product is cetearyl alcohol, a mixture of cetyl alcohol and stearyl alcohol, commercially available under the trade name of LANETTE-O from the company BASF. Cetyl alcohol may also be commercially available under the tradename of LANETTE 16 from the company BASF.

In an embodiment, the solid fatty alcohols of the present invention may be chosen from myristyl alcohol, cetyl alcohol, stearyl alcohol, cetearyl alcohol, and mixtures thereof, octyldodecanol, 2-butyloctanol, 2-hexyldecanol, 2-undecylpentadecanol, oleic alcohol, linoleic alcohol, behenyl alcohol, and mixtures thereof.

Other suitable examples of the solid fatty alcohol of the present invention include branched solid fatty alcohols chosen from 2-dodecylhexadecanol, 2-tetradecyl-1-octadecanol, 2-tetradecyl-1-eicosanol, 2-hexadecyl-1-octadecanol and 2-hexadecyl-1-eicosanol, and mixtures thereof.

In embodiment of the present invention, the fatty alcohol is chosen from cetyl alcohol.

In embodiment of the present invention, the fatty alcohol is chosen from cetearyl alcohol.

In embodiment of the present invention, the fatty alcohol comprises cetyl alcohol and cetearyl alcohol.

In embodiment of the present invention, the fatty alcohol comprises cetyl alcohol and stearyl alcohol.

In embodiment of the present invention, the fatty alcohol is chosen from cetyl alcohol, stearyl alcohol, cetearyl alcohol, and mixtures thereof

In an embodiment, the fatty alcohols of the present invention are chosen from liquid fatty alcohol, solid fatty alcohols, and mixtures thereof.

The fatty alcohol may be present in the composition according to the invention in an amount of from about 1% to 15% by weight, such as from about 1.2% to about 10% by weight, from about 1.4% to about 8% by weight, from about 1.5% to about 5% by weight, or from about 1.5% to about 4% by weight of active material, based on the total weight of the composition, including all ranges and subranges therebetween.

In various embodiments, the total amount of the fatty alcohol is about 1%, 1.25%, 1.5%, 1.75%, 2%, 2.25%, 2.5%, 2.75%, 3%, 3.2%, 3.5%, 3.6%, 3.8%, 4%, 4.25%, 4.5%, 4.75, 5%, 5.5%, 6%, 6.5%, 7%, 7.5%, 8% by weight of active material, based on the total weight of the composition.

### Glyceryl Esters

The glyceryl esters in the compositions of the present invention include, but are not limited to, glyceryl monoesters, such as glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof.

The glyceryl esters in the compositions of the present invention may also be reffered to as nonionic co-emulsifiers.

In one embodiment, the glyceryl ester is chosen from glyceryl oleate, glyceryl monostearate (glyceryl stearate), glyceryl monoisostearate, glyceryl monopalmitate, glyceryl monobehenate, and mixtures thereof.

In one embodiment, the glyceryl ester is chosen from glyceryl stearate.

The at least one glyceryl ester may be present in the composition according to the invention in an amount of from about 0.05% to 5% by weight, such as from about 0.075% to about 4% by weight, from about 0.1% to about 3% by weight, from about 0.2% to about 2% by weight, or from about 0.2% to about 1% by weight of active material, based on the total weight of the composition, including all ranges and subranges therebetween.

In various embodiments, the total amount of the at least one glyceryl ester is about 0.05%, 0.075%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.86%, 0.9%, 0.95%, 1%, 1.25%, 1.5%, 1.75%, 2%, 2.25%, 2.5%, 2.75%, 3%, 3.5%, 4%, 4.5%, 5% by weight of active material, based on the total weight of the composition.

### Organic Acid

The compositions of the present invention comprise at least one organic acid such as mono-carboxylic acids and polycarboxylic acids (with 2 or more carboxylic acid groups).

Suitable examples of the at least one organic acid of the present invention include acetic acid, terephthalic acid, HOOC-PEG-COOH acid; citric acid, tartaric acid; betaine hydrochloride, gluconic acid or 2-ethylcaproic acid, lactic acid, salicylic acid, glycolic acid, malic acid, maleic acid, oxalic acid, malonic acid, aspartic acid, glutamic acid, benzoic acid, acetic acid, formic acid, and mixtures thereof.

Other suitable examples of the at least organic acid are chosen from polycarboxylic acids selected from aspartic acid, glutamic acid, oxalic acid, succinic acid, tartaric acid, mucic acid, citric acid, malic acid, maleic acid, phthalic acid, poly(ethylene glycol) bis(carboxymethyl)ethers, acrylic polyacid, copolymer of acrylic acid and maleic acid, polyaspartic acid, carboxylic polydimethylsiloxanes, and mixtures thereof.

In other embodiments, the organic acid is a sulfonic acid selected from benzene sulfonic acid, sulfonic acid HSO2OH, taurine, 2-[4-(2-hydroxy-ethyl)-piperazin-1-yl]ethane sulfonic acid (or HEPES), and (3E)-3-(4-{(E)-[7,7-dimethyl-3-oxo-4-(sulfomethyl)bicyclo[2.2.1]hept-2-ylidene]methyl}benzylidene)-7,7-dimethyl-2-oxobicyclo[2.2.1]hept-1-yl]methane sulfonic acid, and mixtures thereof.

In an embodiment, the organic acid is selected from trichloroacetic acid, L-poly(ethylene glycol) bis(carboxymethyl)ether having a molecular weight of 250 g/mol, salicylic acid derivatives, jasmonic acid derivative, 3-hydroxy-2-pentyl-cyclopentyl acetic acid, 2-[4-(2-hydroxy-ethyl)-piperazin-1-yl]-ethanesulfonic acid, pyruvic acid, mandelic acid, and mixtures thereof.

In an embodiment, the organic acid is selected from tartaric acid, lactic acid, malic acid, maleic acid, oxalic acid, malonic acid, citric acid, aspartic acid, salicylic acid, benzoic acid, acetic acid, formic acid and mixtures thereof.

In an embodiment, the organic acid is selected from tartaric acid, lactic acid, and mixtures thereof.

The organic acids may be present in a total amount of from about 0.03% to about 3% by weight, such as from about 0.04% to about 2.5% by weight, or such as from about 0.05% to about 2% by weight, or such as from about 0.06% to about 1.5% by weight or such as from about 0.08% to about 1%, based on the total weight of the composition, including all ranges and subranges therebetween.

In one embodiment, the organic acids comprise tartaric acid and lactic acid.

In one embodiment, when the organic acids comprise lactic acid, the lactic acid is present in an amount of from about 0.05% to about 2% by weight, such as from about 0.1% to about 1.5% by weight, or such as from about 0.2% to about 1% by weight, or such as from about 0.25% to about 0.6% by weight, based on the total weight of the composition, including all ranges and subranges therebetween.

In one embodiment, when the at least one organic acid comprises tartaric acid, the tartaric acid is present in an amount of from about 0.03% to about 1% by weight, such as from about 0.04% to about 0.5% by weight, such as from about 0.05% to about 0.3% by weight, based on the total weight of the composition, including all ranges and subranges therebetween.

In one embodiment, the tartaric acid is present an amount such that the ratio by weight of the second cationic surfactant to tartaric acid ranges from about 6 to about 2 or from about 5 to about 2.5 or from about 4.3 to about 2.8, including all ranges and subranges therebetween, and is preferably, at about 3.8.

In one embodiment, the lactic acid is present an amount such that the ratio by weight of APTES to lactic acid ranges from about 3 to about 0.5 or from about 2 to about 0.6 or from about 1.7 to about 0.9, including all ranges and subranges therebetween, and is preferably, at about 1.4.

### Water

The compositions according to various embodiments of the disclosure may be aqueous. Water can be present in amounts of about 95% or less, such as from about 95% to about 5% by weight, or about 90%, 80%, 70%, 60%, 50%, 40%, 30%, 20%, 10%, or 5% or less, by weight, based on the total weight of the composition. In further embodiments, water can be present in an amount of about 95%, such as about 90%, 80%, 70%, 60%, 50%, 40%, 30%, 20%, 10%, or 5%, by weight, based on the total weight of the composition, including all ranges and subranges therebetween. Additionally, water can be present in the compositions of the present disclosure in the amount of from about 20% to about 95% by weight, from about 40% to about 90% by weight, or from about 50% to about 80% by weight, based on the total weight of the compositions.

In other embodiments, water can be present in the compositions of the present disclosure in the amount of at least about 95%, 90%, 80%, 70%, 60%, 50%, 45%, 40%, 30%, 20%, 10%, 5% by weight, based on the total weight of the compositions.

### Second Silane Compound

The compositions of the present disclosure may further comprise a second silane compound chosen from silicones which are compounds other than the first silane compounds described above. These silicones may be chosen from amino silicones and dimethicone.

In an embodiment, the second silane compound of the present disclosure is an amino silicone.

The term “amino silicone” is intended to mean any silicone comprising at least one primary, secondary or tertiary amine or a quaternary ammonium group (i.e., a quaternized group).

As amino silicone that may be used in the scope of the instant disclosure, the following can be cited:

a) polysiloxanes corresponding to formula (A):

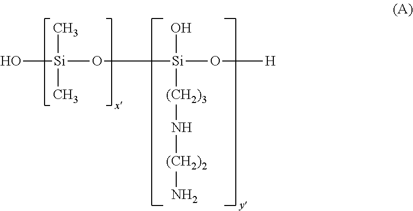

in which x′ and y′ are integers such that the weight-average molecular weight (Mw) is comprised between about 5000 and 500 000;

b) amino silicones corresponding to formula (B):

R′ₐG₃₋ₐ-Si(OSiG₂)ₙ(OSG_{b}R′_{2-b})ₘ-O-SiG₃₋ₐ-R′ₐ (B)

in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula -CqH₂qL in which q is a number ranging from 2 to 8 and L is an optionally quaternized amino group chosen from the following groups:

—NR″-Q-N(R″)₂

—N(R″)₂

—N+(R″)₃A-

—N+H(R″)₂A-

—N+H₂(R″) A-

—N(R″)-Q-N+R″H₂A-

—NR″-Q-N+(R″)₂H A-

—NR″-Q-N+(R″)₃A-,

in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

A group of amino silicones corresponding to this definition (B) is represented by the silicones called “trimethylsilylamodimethicone” having formula (C):

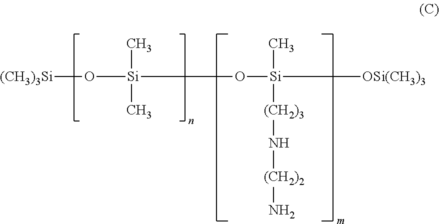

in which n and m have the meanings given above, in formula B.

Another group of amino silicones corresponding to this definition is represented by silicones having the following formulae (D) or (E):

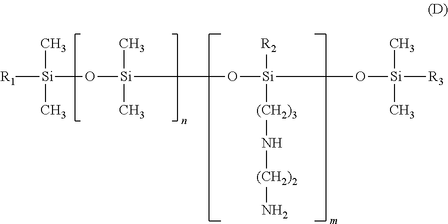

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R₁, R₂, R₃, which may be identical or different, represent a hydroxy or C₁-C₄ alkoxy radical, where at least one of the radicals R₁ to R₃ denotes an alkoxy radical.

The alkoxy radical is preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

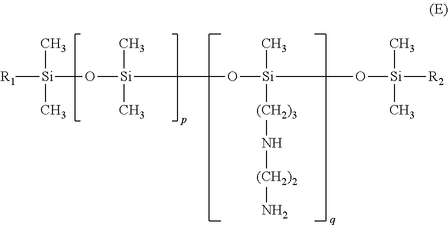

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R₁, R₂, which are different, represent a hydroxy or C₁-C₄ alkoxy radical, where at least one of the radicals R₁ or R₂ denotes an alkoxy radical.

The alkoxy radical is preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name Belsil® ADM 652.

A product containing amino silicones having structure (E) is sold by Wacker under the name Fluid WR 1300@.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino silicones having formula (E), microemulsions are used whose average particle size ranges from 5 nm to 60 nanometres (limits included) and more preferably from 10 nm to 50 nanometres (limits included). Accordingly, according to the invention the microemulsions of amino silicone having formula (E) sold as Finish CT 96 E® or SLM 28020® by Wacker can be used.

Another group of amino silicones corresponding to this definition is represented by the following formula (F):

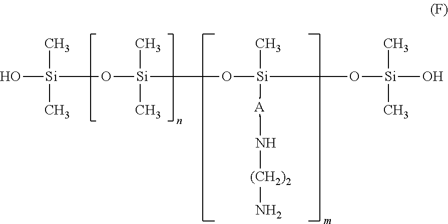

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino silicones corresponding to this definition is represented by the following formula (G):

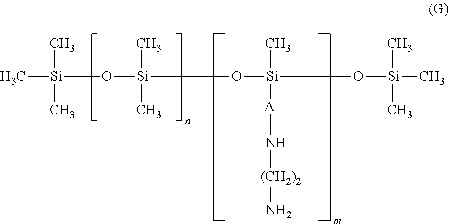

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

A silicone having this formula is for example DC2-8566 Amino Fluid by Dow Corning.

c) amino silicones corresponding to formula (H):

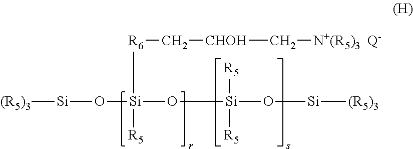

in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C1-C₁₈ alkylene radical or a divalent C1-C₁₈, for example C₁-C₈, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in patent U.S. Pat. No. 4,185,087.

d) quaternary ammonium silicones having formula (I):

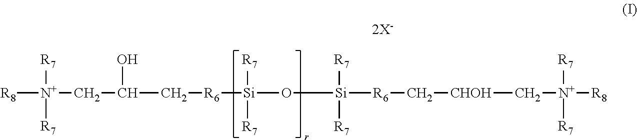

in which:
R₇, which may be identical or different, represent a monovalent hydrocarbon-based radical containing from 1 to 18 carbon atoms, and in particular a C1-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C1-C18, for example C1-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X- is an anion such as a halide ion, in particular chloride, or an organic acid salt (for example acetate);
r represents a mean statistical value from 2 to 200 and in particular from 5 to 100;

These silicones are described, for example, in patent application EP-A 0 530 974.

e) amino silicones having formula (J):

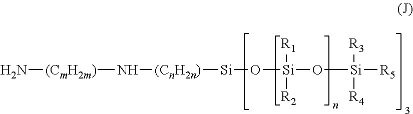

in which:
R₁, R₂, R₃ and R₄, which may be identical or different, denote a C₁-C₄ alkyl radical or a phenyl group;
R₅ denotes a C1-C₄ alkyl radical or a hydroxyl group;
n is an integer ranging from 1 to 5;
m is an integer ranging from 1 to 5;
and in which x is chosen such that the amine number is between 0.01 and 1 meq/g;

f) multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe₂O)xSiMe₂-R—N(R″)—R′—O(C₂H₄O)ₐ(C₃H₆O)_{b}—R′—N(H)—R—]

or alternatively

[—(SiMe₂O)xSiMe₂—R—N(R″)—R′—O(C₂H₄O)ₐ(C₃H₆O)_{b}—]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂-radical; preferentially R denotes a —CH₂CH₂CH₂OCH(OH)CH₂-radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂-radical; preferentially R′ denotes —CH(CH₃)—CH₂-.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names Silsoft™ A-843 or Silsoft™ A+ by Momentive.

g) the alkylamino silicones corresponding to formula (K) below:

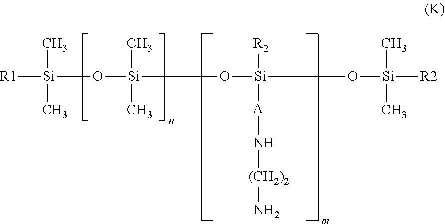

in which:
x and y are numbers ranging from 1 to 5000; preferably, x ranges from 10 to 2000 and especially from 100 to 1000; preferably, y ranges from 1 to 100;
R₁ and R₂, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms,

Preferably, A comprises 3 to 6 carbon atoms, especially 4 carbon atoms; preferably, A is branched. Mention may be made especially of the following divalent radicals: —CH₂CH₂CH₂ and —CH₂CH(CH₃)CH₂—.

Preferably, R₁ and R₂, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R₁ and R₂, which may be identical or different, are chosen from hexadecyl (cetyl) and octadecyl (stearyl) radicals.

Preferentially, the silicone is of formula (K) with:
x ranging from 10 to 2000 and especially from 100 to 1000;
y ranging from 1 to 100;
A comprising 3 to 6 carbon atoms and especially 4 carbon atoms; preferably, A is branched; and more particularly A is chosen from the following divalent radicals: CH₂CH₂CH₂ and —CH₂CH(CH₃)CH₂-; and
R₁ and R₂, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R₁ and R₂, which may be identical or different, being chosen from hexadecyl (cetyl) and octadecyl (stearyl) radicals.

A preferred silicone of formula (K) is bis-cetearylamodimethicone (INCI name).

Mention may be made especially of the silicone sold under the name Silsoft™ AX by Momentive.

h) silicone compounds with at least one quaternary ammonium group.

Suitable non-limiting examples are quaternium 80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium 80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. Most preferred are quaternium 80, silicone quaternium-16, silicone quaternium-18, silicone quaternium-15, and mixtures thereof.

The silicone compounds with at least one quaternary ammonium group can also include those compounds of formula (B) when L in formula (B) is a quaternized amino group as described.

The second silane compound of the present disclosure may be provided or may be commercially available in emulsion form that further comprises surfactants chosen from nonionic surfactants, cationic surfactants, and mixtures thereof. In certain embodiments, the emulsion in which the second silane is contained is a microemulsion.

Preferably, the second silane compound chosen from amino silicones are selected from the amino silicones comprising at least one primary, secondary or tertiary amine and from silicone compounds having at least one quaternary ammonium group, and mixtures thereof. Preferred amino silicones comprising at least one primary, secondary or tertiary amine are chosen from amodimethicone (INCI name) sold under the tradename SILSOFT 253, by Momentive Performance Materials or under the tradename XIAMETER® MEM-8299 Cationic Emulsion by Dow Corning or under the tradename Dow Corning 2-8566 Amino Fluid by Dow Corning, bis-isobutyl/PEG/PPG-20/35/amodimethicone copolymer, bis-cetearyl amodimethicone, bis-amino PEG/PPG-41/3 aminoethyl PG-propyl dimethicone (e.g., Silsoft A-843), PEG-40/PPG-8 methylaminopropyl hydroxypropyl dimethicone copolymer (e.g., Silsoft A+), bis-isobutyl/PEG/PPG-20/35/amodimethicone copolymer (e.g., Dow Corning CE 8401 Emulsion), or mixtures thereof. A preferred silicone compound with at least one quaternary ammonium group is silicone quaternium-16 (INCI name) sold under the tradename DOW CORNING or 5-7113 SILICONE QUAT MICROEMULSION, supplied by Dow Corning.

The material or emulsion containing the second silane compound(s) may be present in the composition according to the invention in an amount of about 0.04% to about 1% by weight, such as from about 0.1% to about 0.8% by weight, from about 0.1% to about 0.7% by weight, from about 0.15% to about 0.6% by weight, or from about 0.2% to about 0.5%, by weight of the material or emulsion, based on the total weight of the composition, including all ranges and subranges therebetween.

In various embodiments, the total amount of the material or emulsion containing the second silane compound is about 0.01%, 0.025%, 0.04%, 0.05%, 0.075%, 0.1%, 0.15%, 0.2%, 0.25%, 0.3%, 0.33%, 0.35%, 0.4%, 0.44%, 0.45%, 0.5%, 0.55%, 0.6%, 0.65%, 0.7%, 0.75%, 0.8%, 0.85%, 0.88%, 0.9%, 0.95%, 1%, 1.25%, 1.5%, 1.75%, and 2% by weight of the material or emulsion, based on the total weight of the composition.

pH

The pH of the compositions according to the disclosure generally ranges from about 3 to less than 7, for example, from about 3.1 to about 6.5, or from about 3.2 to about 6.0, or from about 3.4 to about 5.5, preferably from about 3.5 to about 5, and more preferably from about 3.5 to about 4.5, such as from about 3.5 to about 4.4 or such as from about 3.6 to about 4.3, including ranges and subranges therebetween. In certain embodiments, the pH of the compositions according to the disclosure is at about 3, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.2, 6.4, 6.6, 6.8, and 7.

### Additional Components

The composition according to the disclosure may also comprise additives chosen from cationic polymers other than the claimed cationic compounds, nonionic polymers, rheology modifiers, thickening and/or viscosity modifying agents, associative or non-associative polymeric thickeners, non-polymeric thickeners, non-polymeric surfactants (nonionic, cationic or amphoteric), nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preservatives such as benzyl alcohol, phenoxyethanol, sorbic acid, methyl paraben, propyl paraben, methylchloroisothiazolinone, methylisothiazolinone, pH stabilizers, alkaline neutralizing agents, organic solvents (e.g., alcohols, polyols, silicone oils, and hydrocarbons), and mixtures thereof. A person skilled in the art will take care to select the optional additives and the amount thereof such that they do not harm the properties of the compositions of the present disclosure.

If present in the composition, these additives are generally present in an amount ranging up to about 40% by weight of active material relative to the total weight of the composition, such as up to about 30%, up to about 20%, up to about 15%, up to about 10%, up to about 5%, such as from 0% to 20%.

The compositions of certain embodiments may comprise stabilizers, for example sodium chloride, magnesium dichloride or magnesium sulfate.

The rheology modifiers and thickening/viscosity-modifying agents that may be employed in compositions of the present disclosure may include any water-soluble or water-dispersible compound that is compatible with the compositions of the disclosure, such as acrylic polymers, non-acrylic polymers, starch, cellulose-based polymers, non-polymeric and polymeric gelling agents, and mixtures thereof.

The compositions may be packaged in various forms, especially in bottles, in pump bottles or in tubes or in jars or in aerosol containers so as to apply the composition in vaporized form or in the form of a mousse. The compositions may also impregnate applicators, especially gloves or wipes.

The composition may be applied by hand, with an applicator nozzle, with a container equipped with a pump and a dispensing comb, or with an insoluble substrate impregnated with the composition.

### Processes/Methods

The compositions according to the disclosure may be prepared according to techniques that are well known to those skilled in the art.

Embodiments of the disclosure also relate to a process for treating keratinous materials, such as hair, which consists in applying an effective amount of a composition as defined above to the said keratinous materials, and in rinsing, for example with water, after an optional leave-on time.

Certain embodiments also relate to a process for conditioning keratinous materials, which consists in applying an effective amount of a composition as defined above to the said keratinous materials, and in optionally rinsing, for example with water, after an optional leave-on time.

In some embodiments, keratinous materials, such as hair, may be washed or cleansed by a first step of applying a shampoo or cleansing or detergent-based composition, with an optional leave-on time, followed by a second step of applying the composition of the disclosure onto hair, with an optional step of rinsing the hair with water between the two first and second steps, and optionally rinsing the composition of the disclosure, for example with water, after an optional leave-on time.

The compositions may be applied to keratinous substrates, such as the hair, and subsequently rinsed off. In various embodiments, the compositions comprise hair care compositions for conditioning the hair, and in various embodiments the hair care composition will traditionally be rinsed off the hair within a short period of time after application to the hair, such as a period of time up to about 10 minutes, up to about 5 minutes, or up to about 2 minutes after application to the hair.

In various embodiments, processes according to the disclosure comprise applying the compositions described onto keratinous substrates, such as the hair, and subsequently rinsing the compositions off. The processes may, in various embodiments, impart conditioning and/or volume to the keratinous substrate to which the composition is applied, even after the composition is rinsed off. The processes may additionally impart long lasting volume as well as conditioning to the keratinous substrates.

As used herein, the method/process and composition disclosed herein may be used on the hair that has not been artificially dyed, pigmented or permed.

As used herein, the method/process and composition disclosed herein may be also used on the hair that has been artificially dyed, pigmented or permed.

### EXAMPLES

The following Examples are intended to be non-restrictive and explanatory only, with the scope of the disclosure being defined by the claims.

The ingredient amounts in the compositions/formulas described below are expressed in % by weight, based on the total weight of the composition/formula.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result.

### Example 1: Compositions

TABLE 1
Conditioner Compositions

| | FORMULAS | | |
|---|---|---|---|
| US INCI NAME | Formula A | Formula B | Formula C |
| BEHENTRIMONIUM CHLORIDE | 1.32 | 0.82 | 0.82 |
| STEARAMIDOPROPYL | 0.33 | 0.83 | 0.83 |
| DIMETHYLAMINE | | | |
| AMINOPROPYL | 0.5 | 0.5 | 0.5 |
| TRIETHOXYSILANE | | | |
| GLYCERYL STEARATE | 0.86 | 0.86 | 0.86 |
| CETYL ALCOHOL | 1.79 | 1.79 | 1.79 |
| CETEARYL ALCOHOL | 1.79 | 1.79 | 1.79 |
| POLYQUATERNIUM-11 | 0.1 | 0.1 | 0.1 |
| TARTARIC ACID | 0.087 | 0.22 | 0.22 |
| LACTIC ACID | 0.36 | 0.36 | 0.36 |
| SILICA SILYLATE | — | 0.2 | — |
| POLYQUATERNIUM-37 (and) | 0.1 | — | 0.1 |
| PROPYLENE GLYCOL | | | |
| DICAPRYLATE/DICAPRATE | | | |
| (and) PPG-1 TRIDECETH-6 | | | |
| AMODIMETHICONE (and) | 0.5 | 0.25 | — |
| C11-15 PARETH-7 (and) | | | |
| LAURETH-9 (and) GLYCERIN | | | |
| (and) TRIDECETH-12 | | | |
| AUXILIARY INGREDIENTS | 1.0 | 0.9 | 0.9 |
| (e.g., glycerin, organic solvent, | | | |
| fragrance) | | | |
| phenoxyethanol | 0.7 | 0.7 | 0.7 |
| benzyl alcohol | 0.004 | 0.002 | — |
| WATER | QS 100 | QS 100 | QS 100 |

Formulas above were prepared according to Table 1 and the following protocol.

Preparation of Side Kettle:
1. For formulas A and C, 5% by weight of the total rest of the water was mixed with Polyquaternium-37 (and) Propylene glycol dicarprylate/Dicaprate (and) PPG-1 Trideceth-6 in a container.
2. In a separate container, Aminopropyl Triethoxysilane (APTES) was mixed with another 7% by weight of the total water and neutralized with the appropriate weight of lactic acid to a pH of about 4.
3. The mixtures in 1 and 2 were combined and mixed until homogeneous.
4. For formula B, only step 2 was followed.

### Preparation of Main Kettle

5. The rest of the water was mixed with Polyquaternium-11 in a container.
6. Stearmidopropyl dimethylamine was added and the resulting mixture was mixed and heated to about 70° C.
7. The pH of the mixture was adjusted with the appropriate weight of Tartaric acid.
8. Behentrimonium chloride was added to the main kettle.
9. Fatty alcohol (e.g., cetearyl alcohol and cetyl alcohol), and glyceryl stearate were added and the mixture was homogenize for about 10-15 minutes.
10. The mixture was cooled to about 50° C. and auxiliary ingredients such as preservatives, fragrance were added; a silicone (e.g., amodimethicone emulsion), if present, and/or silica silylate, if present, were also added at this step.
11. The mixture was further cooled to room temperature
12. The contents of the side kettle were added to the main kettle and mixed with the rest of the mixture.

The final formulas had a pH within the range of 3.5-4.5, preferably at 4.0. To achieve this pH range, the ratio by weight of APTES to lactic acid was within the range of from about 1.7 to about 0.9, such as at about 1.4 and the ratio by weight of stearmidopropyl dimethylamine to tartaric acid was within the range of from about 4.3 to about 2.8, such as at about 3.8.

### Example 2: Comparative Commercial Product

TABLE 2
Comparative Conditioner Formula Y
(advertised for its volumizing properties)
US INCI NAME
Water
Cetyl Alcohol
Steramidopropyl Dimethylamine
Stearyl Alcohol
Quaternium 18
Fragrance
Bis Aminopropyl Dimethicone
Benzyl Alcohol
Cetearyl Alcohol
Hydroxypropyl Guar
Glyceryl Stearate
Panthenyl Ethyl Ether
Oleyl Alcohol
Trisodium Ethylenediamine
Methylchloroisothiazolinone
Methylsothiazolinone

### Example 3 Testing on Hair—Salon Expert Evaluation and Consumer Tests

The performance/effect of the inventive conditioner on hair was evaluated in a shampoo-conditioner regimen and compared with the performance of a shampoo-conditioner regimen using the comparative commercial conditioner product above. The shampoos used in the shampoo-conditioner regimens mainly comprise anionic sulfate surfactants and amphoteric surfactants.

In a consumer test of 60 panelists, in comparison to the commercial product, Formula A performed comparably on the hair on the attributes of amount of volume, fuller look to the hair, amount of body to the hair, conditioned feel, ease of combing, and ease of styling/shaping the hair. The ratings for Formula A were trending higher on the attributes of ease of detangling, nourished feel, ease of blow-drying the hair, more pleasant to use and overall satisfaction

In an expert evaluation study using Formula B on the hair of 8 human panelists, Formula B was found to provide similar level of conditioning as the commercial conditioner product at the wet state with slight better wet bounce and less weight. In the dry state, Formula B provided better final volume, slightly less weight and slightly more body to the hair.

In a separate consumer test of 31 panelists, Formula B performed better on the hair on the attributes of amount of volume, full hair feel, and acceptable amount of bounce and performed comparably on the attributes of fuller look to the hair and root lift.

In consumer tests on 15 panelists, formula C tested slightly better than commercial conditioner product in volume and fuller appearance. Formula C also provided slightly more moisturization and weighed down the hair less.

A separate expert evaluation test also showed that formula C provided more body to the hair in dry state (although the hair was less supple) and smooth feel to the hair in the wet state.

It is to be understood that the foregoing describes preferred embodiments of the disclosure and that modifications may be made therein without departing from the spirit or scope of the disclosure as set forth in the claims.

What is claimed is:

1. A composition for treating keratinous substrates, the composition comprising:
(a) at least one first cationic surfactant chosen from alkyl quaternary ammonium or diammonium salts, and mixtures thereof;
(b) at least one a second cationic surfactant chosen from alkyl amines, alkyl amine salts, and mixtures thereof;
(c) a first silane compound corresponding to formula (I):
R₁Si(OR₂)_{z}(R₃)ₓ(OH)_{y} (I)
in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3;
(d) at least one cationic vinylpyrrolidone polymer;
(e) two or more fatty alcohols;
(f) at least one glyceryl ester;
(g) organic acids; and
(h) water.

2. The composition according to claim 1, wherein the first cationic surfactant is chosen from alkyl quaternary ammonium, and mixtures thereof.

3. The composition according to claim 2, wherein the first cationic surfactant is chosen from behentrimonium chloride, cetrimonium chloride, quaternium-22, behenylamidopropyl-2,3-di-hydroxypropyldimethylammonium chloride, palmitylamidopropyltrimethylammonium chloride, and chloride and methyl sulfate of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, distearyldimethylammonium chloride, oleocetyldimethylhydroxyethylammonium chloride, stearamidopropyldimethyl (myristyl acetate) ammonium chloride, di(C1-C2 alkyl) (C12-C22 alkyl)hydroxy(C1-C2alkyl)ammonium salt, or alkyltrimethylammonium salt in which the alkyl radical comprises 12 to 24 carbon atoms, propanetallowdiammonium dichloride, behentrimonium methosulfate, and mixtures thereof.

4. The composition according to claim 1, wherein the amount of the first cationic surfactant ranges from about 0.1% to about 10%, based on the total weight of the composition.

5. The composition according to claim 1, wherein the second cationic surfactant is chosen from dimethylamine derivatives.

6. The composition according to claim 5, wherein the dimethylamine derivative is chosen from stearyl dimethyl amine, stearamidopropyl dimethylamine, brassicamidopropyl dimethylamine, and mixtures thereof.

7. The composition according to claim 1, wherein the amount of the second cationic surfactant ranges from about 0.05% to about 5% by weight, based on the total weight of the composition.

8. The composition according to claim 1, wherein the first silane compound (c) of formula (I) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, their oligomers, and mixtures thereof.

9. The composition according to claim 1, wherein the amount of the first silane compound (c) of formula (I) ranges from about 0.05% to about 5% by weight, based on the total weight of the composition.

10. The composition according to claim 1, wherein the cationic vinylpyrrolidone polymer is chosen from vinylpyrrolidone/dimethylaminoethyl methacrylate copolymers quaternized with diethyl sulfate, polymeric quaternary ammonium salt consisting of vinylpyrrolidone and dimethylaminopropyl methacrylamide monomers, and mixtures thereof.

11. The composition according to claim 1, wherein the amount of the cationic vinylpyrrolidone polymer ranges from about 0.05% to about 5% by weight, based on the total weight of the composition.

12. The composition according to claim 1, wherein the two or more fatty alcohols are chosen from non-alkoxylated, saturated or unsaturated, linear or branched fatty alcohols having from 6 to 60 carbon atoms.

13. The composition according to claim 1, wherein the two or more fatty alcohols are chosen cetyl alcohol, stearyl alcohol, cetearyl alcohol (mixture of cetyl alcohol and stearyl alcohol), octyldodecanol, 2-butyloctanol, 2-hexyldecanol, 2-undecylpentadecanol, oleic alcohol, linoleic alcohol, behenyl alcohol, 2-dodecylhexadecanol, 2-tetradecyl-1-octadecanol, 2-tetradecyl-1-eicosanol, 2-hexadecyl-1-octadecanol and 2-hexadecyl-1-eicosanol, octyldodecanol, 2-butyloctanol, 2-hexyldecanol, 2-undecylpentadecanol, oleyl alcohol or linoleyl alcohol, isostearyl alcohol, and mixtures thereof.

14. The composition according to claim 1, wherein the amount of the fatty alcohols ranges from about 1% to about 15% by weight, based on the total weight of the composition.

15. The composition according to claim 1, wherein the glyceryl ester is chosen from glyceryl monoesters of C16-C22 saturated, unsaturated and branched chain fatty acids, and mixtures thereof.

16. The composition according to claim 1, wherein the glyceryl ester is chosen from glyceryl oleate, glyceryl monostearate (or glyceryl stearate), glyceryl monoisostearate, glyceryl monopalmitate, glyceryl monobehenate, and mixtures thereof.

17. The composition according to claim 1, wherein the amount of the gylceryl ester ranges from about 0.05% to about 5% by weight, based on the total weight of the composition.

18. The composition according to claim 1, wherein the organic acids are chosen from mono-carboxylic acids and polycarboxylic acids (with 2 or more carboxylic acid groups).

19. The composition according to claim 1, wherein the organic acids are chosen from acetic acid, terephthalic acid, HOOC-PEG-COOH acid; citric acid, tartaric acid; betaine hydrochloride, gluconic acid or 2-ethylcaproic acid, lactic acid, salicylic acid, glycolic acid, malic acid, maleic acid, oxalic acid, malonic acid, aspartic acid, glutamic acid, benzoic acid, acetic acid, formic acid, aspartic acid, glutamic acid, oxalic acid, succinic acid, mucic acid, phthalic acid, poly(ethylene glycol) bis(carboxymethyl)ethers, acrylic polyacid, copolymer of acrylic acid and maleic acid, polyaspartic acid, and carboxylic polydimethylsiloxanes, sulfonic acids such as benzene sulfonic acid, sulfonic acid HSO2OH, taurine, 2-[4-(2-hydroxy-ethyl)-piperazin-1-yl]ethane sulfonic acid (or HEPES), and (3E)-3-(4-{(E)-[7,7-dimethyl-3-oxo-4-(sulfomethyl)bicyclo[2.2.1]hept-2-ylidene]methyl}benzylidene)-7,7-dimethyl-2-oxobicyclo[2.2.1]hept-1-yl]methane sulfonic acid, trichloroacetic acid, L-poly(ethylene glycol) bis(carboxymethyl)ether having a molecular weight of 250 g/mol, salicylic acid derivatives, jasmonic acid derivative, 3-hydroxy-2-pentyl-cyclopentyl acetic acid, 2-[4-(2-hydroxy-ethyl)-piperazin-1-yl]-ethanesulfonic acid, pyruvic acid, mandelic acid, and mixtures thereof.

20. The composition according to claim 1, wherein the amount of the organic acid ranges from about 0.03% to about 3% by weight, based on the total weight of the composition.

21. The composition according to claim 20, wherein the tartaric acid is present an amount such that the ratio by weight of the second cationic surfactant to tartaric acid ranges from about 6 to about 2.

22. The composition according to claim 20, wherein the lactic acid is present an amount such that the ratio by weight of APTES to lactic acid ranges from about 3 to about 0.5.

23. The composition according to claim 1, further comprising a second silane compound other than the first silane (c) and chosen from dimethicone, amino silicones comprising at least one primary, secondary or tertiary amine or a quaternary ammonium group, and mixtures thereof.

24. The composition according to claim 23, wherein the amount of the second silane compound ranges from about 0.04% to about 1% by weight, based on the total weight of the composition.

25. The composition according to claim 23, wherein the second silane compound is chosen from amino silicones.

26. The composition according to claim 25, wherein the amino silicone is provided as an emulsion comprising at least one surfactant selected from a nonionic surfactant, a cationic surfactant, and mixtures thereof.

27. The composition according to claim 1, wherein the keratinous substrates include hair.

28. The composition according to claim 1, wherein the composition is a rinse-off conditioning composition.

29. The composition according to claim 1, wherein the composition is capable of being applied onto the keratinous substrates following the application of a shampoo or a cleansing or a detergent-based composition onto the keratinous substrates.

30. A composition for conditioning hair, the composition comprising:
(a) from about 0.1% to about 10% by weight of at least one first cationic surfactant chosen from alkyl quaternary ammonium or diammonium salts, and mixtures thereof;
(b) from about 0.05% to about 5% by weight of at least one second cationic surfactant chosen from dimethylamine derivatives;
(c) from about 0.05% to about 5% by weight of a first silane compound corresponding to formula (I):
R₁Si(OR₂)_{z}(R₃)ₓ(OH)_{y} (I)
in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C1-C₂₀, in particular C1-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3;
(d) from about 0.05% to about 5% by weight of at least one cationic vinylpyrrolidone polymer chosen from vinylpyrrolidone/dimethylaminoethyl methacrylate copolymers quaternized with diethyl sulfate, polymeric quaternary ammonium salt consisting of vinylpyrrolidone and dimethylaminopropyl methacrylamide monomers, and mixtures thereof;
(e) from about 1% to about 15% by weight of fatty alcohols chosen from cetyl alcohol, stearyl alcohol, cetearyl alcohol (mixture of cetyl alcohol and stearyl alcohol), octyldodecanol, 2-butyloctanol, 2-hexyldecanol, 2-undecylpentadecanol, oleic alcohol, linoleic alcohol, and mixtures thereof;
(f) from about 0.05% to about 5% by weight of at least one glyceryl ester;
(g) organic acids chosen from tartaric acid, lactic acid, malic acid, maleic acid, oxalic acid, malonic acid, citric acid, aspartic acid, salicylic acid, benzoic acid, acetic acid, formic acid, and mixtures thereof; and
(h) water;
all weights being based on the total weight of the composition.

31. A method for treating and/or conditioning a keratinous substrate, the method comprising contacting the keratinous substrate with a composition comprising the composition of claim 1.

32. The method according to claim 31, wherein the keratinous substrate is hair.

33. The method according to claim 31, further comprising leaving the composition on the keratinous substrate for a leave-on time and then rinsing the keratinous substrate with water.

34. A multi-component hair cosmetic agent for cleansing and conditioning hair, the hair cosmetic agent comprising:
a) a first component comprising a shampoo or a cleansing or a detergent-based composition: and
b) a second component comprising the composition of claim 1;
wherein the first and second components are each packaged in a separate packaging assembly.
